(19) United States
(12) Patent Application Publication
(10) Pub. No.: US 2009/0082796 A1
(43) Pub. Date: Mar. 26, 2009

(54) MODIFIED TENOTOMY SCISSORS WITH CONJUNCTIVA RISERS

(75) Inventors: WERHNER C. ORILLA, Anaheim, CA (US); Michael R. Robinson, Irvine, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/859,639

(22) Filed: Sep. 21, 2007

(51) Int. Cl. A61B 17/3201; B26B 13/00

(52) U.S. Cl. 606/167; 30/254

(57) ABSTRACT

A tenotomy scissors for implantation procedures generally includes a first blade having a handle and a tip with a cutting edge thereon, a second blade having a handle and a tip with a cutting edge thereon, along with a swivel pivotally attaching the first and second blades for enabling insertion of the cutting edges for cutting a pocket in tissue when the handle is operated. One or more risers upstanding from at least one of the blade tips are provided for keeping the pocket open to facilitate insertion of an implant device.

The present invention is related to new and useful improvements in surgical procedures and apparatus for opening a space, cavity, or pocket in tissue.

Heretofore, device implementation procedures typically have utilized tenotomy scissors to provide a sub-conjunctival sub-tenon access into tissue by forming a pocket with a flap thereover.

A forceps is utilized to keep the flap open in order to insert the implant into the pocket. This procedure requires two surgeons to conduct the implantation.

The present invention will allow but one surgeon to conduct a sub-conjunctival/sub-tenon implantation procedure and consequently provides a great benefit for single practitioner ophthalmology clinics.

### SUMMARY OF THE INVENTION

A tenotomy scissors in accordance with the present invention generally includes a first blade having a handle and a tip with a cutting edge thereon and a second blade having a handle and a tip with a cutting edge thereon.

A swivel is provided which pivotally attaches the first and second blades for enabling intersection of the cutting edges for cutting a pocket and tissue when the handle is operated.

At least one riser is provided which upstands from at least one of the blade tips and is disposed in a spaced apart relationship with a corresponding cutting edge for keeping the pocket open to facilitate insertion of an implant device.

More particularly, the scissors may include lateral elements which extends toward the corresponding cutting edge and the riser upstands from a tip edge opposite the cutting edge.

A sloped leading edge may be provided on the riser for facilitating entry of the riser into the created pocket.

In one embodiment, a second riser may be provided which upstands from another of the blade tips and is disposed in a spaced apart relationship with a corresponding cutting edge.

A method for implanting a device and tissue utilizing the scissors of the present invention generally includes creating a pocket using the cutting edges, keeping the pocket open by using the riser, and inserting the device.

### BRIEF DESCRIPTION OF THE DRAWINGS

The advantages and features of the present invention will be better understood by the following description when considered in conjunction with the accompanying drawings, of which:

FIG. 1 is a perspective view of the scissors in accordance with the present invention generally showing a first blade with a handle and a tip with a cutting edge, a second blade having a handle a tip with a cutting edge, the blades being pivotally attached to one another and each including an upstanding riser from each of the blades;

FIG. 2 is an illustration of the use of the scissors shown in FIG. 1 showing operation of the scissor risers for keeping a pocket open while an implant is inserted;

FIG. 3 is a perspective view of another embodiment to the present invention similar to that shown in FIG. 1 but with only one riser including a lateral roof extending therefrom; and

FIG. 4 illustrates the use of the scissors shown in FIG. 3.

### DETAILED DESCRIPTION

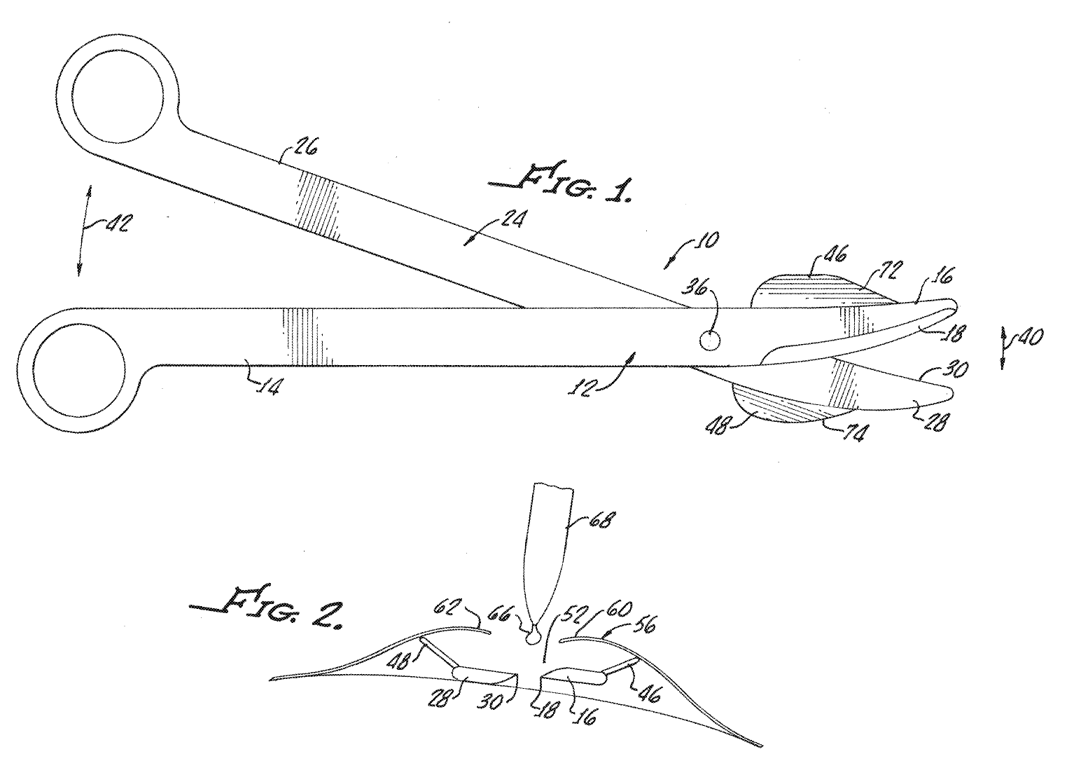

With reference to FIG. 1, there is shown tenotomy scissors 10 in accordance with the present invention generally including a first blade 12 including handle 14 and a tip 16 with a cutting edge 18 thereon along with a second blade 24 including a handle 26, tip 28 with a cutting edge 30 thereon.

A swivel 36 pivotally attaches the first and the second blades 12, 24 for enabling intersection of the cutting edges for cutting purposes as indicated by the arrow 40 as the handles 14, 26 are operated as indicated by the arrow 42. Risers 46, 48 attached to or formed with the blade tips 16, 28 respectively upstand therefrom and are disposed in a spaced apart relationship with corresponding cutting edges 18, 30. The blades and risers may be formed from any suitable material such as titanium.

In operation, as illustrated in FIG. 2, after a pocket 52 has been established under a conjunctival epithelium 56 spreading of the risers 46, 48 keep epithelium flaps 60, 62 separated to facilitate the insertion of an implant 66 by an instrument 68.

Since the scissors 10 is operated with one hand of a surgeon, the surgeon can manipulate the instrument 68 with another hand thus eliminating the need for an assisting surgeon.

With regard to establishing the pocket, sloped leading edges 72, 74 facilitate entry of the risers into the pocket 52.

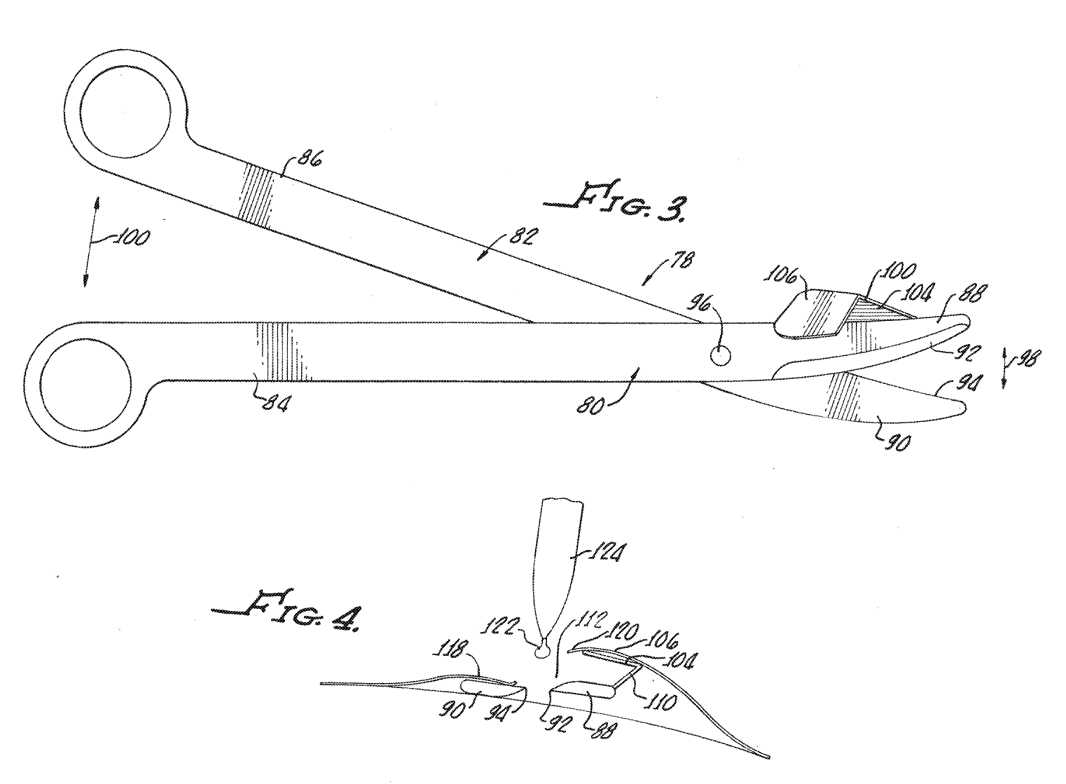

Another embodiment of the scissors 78 is shown in FIG. 3. Similar to the scissors 10 shown in FIG. 1, the scissors 78 include blades 80, 82 with handles 84, 86 along with corresponding tips 88, 90 and cutting edges 92, 94 a swivel 96 pivotally attaches the blades 80, 82 for enabling intersection of the cutting edges 92, 94 indicated by the arrow 98 by operation of the handles 84, 86 as indicated by the arrow 100. The scissors 78 includes one riser 104 upstanding from the tip 88 and includes a roof, or a lateral element 106 extending over the corresponding cutting edge 92. Similar to the risers 72, 74 of the scissors 10 the riser 104 includes a sloped leading edge 110.

With reference to FIG. 4, an operation after establishing a pocket beneath the conjunctival epithelium 114, the riser 104 with roof 106, along with the blade tip 90, keep the pocket 112 open by separating flaps 118, 120 to facilitate the implantation of a device 122 by an instrument 124, as hereinabove described in connection with the scissors 10 shown in FIGS. 1 and 2.

It follows that a method in accordance with the present invention for implanting a device in tissue using the scissors 10, 78 includes creating the pocket 52, 112 using the cutting edges 18, 20, 92, 94, keeping the pocket 52, 112 open using the risers 46, 48, 104 and inserting the device 122.

Although there has been hereinabove described a specific modified tenotomy scissors with conjunctiva risers and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A tenotomy scissors comprising:
a first blade having a handle and a tip with a cutting edge thereon;
a second blade having a handle and a tip with a cutting edge thereon;
a swivel pivotally attaching the first and second blades for enabling intersection of the cutting edges for cutting purposes when the handles are operated; and
a riser upstanding from at least one of the blade tips and disposed in a spaced apart relationship with a corresponding cutting edge.

2. The scissors according to claim 1 wherein said riser includes a lateral element extending toward the corresponding cutting edge.

3. The scissors according to claim 1 wherein said riser upstands from a tip edge opposite the corresponding cutting edge.

4. The scissors according to claim 1 wherein said riser includes a sloped leading edge.

5. The scissors according to claim 1 further comprising a second riser upstanding from another of the blade tips and disposed in a spaced apart relationship with a corresponding cutting edge.

6. The scissors according to claim 5 wherein each riser upstands from a tip edge opposite the corresponding cutting edge.

7. The scissors according to claim 6 wherein each riser includes a sloped leading edge.

8. A tenotomy scissors for implantation procedures, the scissors comprising:
a first blade having a handle and a tip with a cutting edge thereon;
a second blade having a handle and a tip with a cutting edge thereon;
a swivel pivotally attaching the first and second blades for enabling intersection of the cutting edges for cutting a pocket in tissue when the handle an operated; and
a riser upstanding from at least one of the blade tips and disposed in a spaced apart relationship with a corresponding cutting edge for keeping the pocket open to facilitate insertion of an implant device.

9. The scissors according to claim 8 wherein said riser includes a lateral element extending over the corresponding cutting edge.

10. The scissors according to claim 8 wherein said riser upstands from a tip edge opposite the corresponding cutting edge.

11. The scissors according to claim 8 wherein said riser includes a sloped leading edge.

12. The scissors according to claim 8 further comprising a second riser upstanding from another of the blade tips and disposed in a spaced apart relationship with a corresponding cutting edge.

13. The scissors according to claim 12 wherein each riser upstands from a tip edge opposite the corresponding cutting edge.

14. The scissors according to claim 13 wherein each riser includes a sloped leading edge.

15. A method for implanting a device in tissue using the scissors of claim 1 said method comprising:
creating a pocket using the cutting edges;
keeping the pocket open using the riser; and
inserting the device.
